(19) United States
(12) Patent Application Publication
(10) Pub. No.: US 2006/0116608 A1
(43) Pub. Date: Jun. 1, 2006

(54) Catheter feed through guide

(76) Inventors: Andrew I Poutiatine, San Anselmo, CA (US); James A Filice, Los Gatos, CA (US)

Correspondence Address: DURECT CORPORATION, 10240 BUBB ROAD, CUPERTINO, CA, 95014, US

(21) Appl. No.: 10/537,443

(22) PCT Filed: Dec. 4, 2003
(86) PCT No.: PCT/US03/38448

§ 371 Date: Jun. 2, 2005

Related U.S. Application Data
(60) Provisional application No. 60/430,866, filed on Dec. 4, 2002.

(51) Int. Cl. A61M 25/00

(52) U.S. Cl. 600/585

(57) ABSTRACT

A catheter feed through guide apparatus and method which facilitates the feeding of a catheter through a tunneler during implant procedures and avoids damage to the catheter and catheter tip during the catheter feed through procedure. The apparatus comprises a guide element configured to fit through a tunneler and to releasibly engage a catheter or catheter end.

### FIELD OF THE INVENTION

This invention pertains generally to implantable drug delivery systems, and more particularly to a catheter feed through guide apparatus and method which facilitates the placement of catheter through a tunneler during implant procedures

### BACKGROUND OF THE INVENTION

Implantable drug delivery devices are increasingly used as therapeutic tools for treatment of a variety of conditions and diseases, especially where a prolonged period of therapy is required. Implantable drug delivery devices avoid patient inconvenience and discomfort associated with administration of multiple doses of an agent, and further provide for enhanced therapeutic benefits due to avoidance of bolus doses, improved patient compliance with dosage regimens, and providing generally a constant blood serum level of delivered drugs.

Various implantable drug delivery systems have been developed using different technologies to accomplish movement of drug (typically in a drug formulation, e.g., a solvent) from within a reservoir in the device through an exit port or orifice to a treatment site in the subject. These delivery technologies have been based on, inter alia, diffusive, erodible, and convective mechanisms. Exemplary delivery systems employing convection include, but are not limited to, electromechanical pumps, osmotic pumps, electro-osmotic pumps, electro-chemical pumps, hydrolytic systems, piezoelectric pumps, elastomeric pumps, vapor pressure pumps, and electrolytic pumps.

The location of a specific drug delivery site in the body often will be non-optimal for locating an implantable pump. This situation occurs, for example, in the delivery of hydromorphone to the spinal column. In such instances the pump is implanted remotely from the actual drug delivery site and the is drug transported from the pump to the delivery site via an implanted catheter, with one end of the catheter coupled to the pump, and the other end delivering the drug from the pump to the selected site. The implanted catheter may traverse a substantial distance between the pump location and the drug delivery site, and a tunneler device is usually employed to facilitate the implantation of the catheter.

The tunneler is typically a long tubular device made of stainless steel or polymeric material. Incisions are made in a patient adjacent to the pump location and the drug delivery site, and an end of the tunneler is inserted in one incision and passed under the patient's skin until it reaches the other incision. The tunneler thus defines a path under the skin for the catheter between the incisions. A catheter end is then inserted into the opening at one of the tunneler ends, and the catheter is passed through the tunneler until the catheter arrives at the opposite end of the tunneler. The tunneler is then removed to leave the catheter in place beneath the skin. The delivery tip of the catheter is positioned at the drug delivery site, and the opposite end is coupled to the drug pump and the pump is sutured in place within its incision, after which the incisions are closed.

The tunneler is relatively thin to facilitate its passage under the skin and to minimize discomfort to the patient associated with the implant procedure. FIG. 1 illustrates generally the prior art manner of introducing a flexible elongated catheter 10 into the bore 12 of a tubular tunneler element 14. The diameter of bore 12 and tunneler 14 are exaggerated for clarity. The catheter tip 16 is manually inserted into bore 12, and the catheter 10 is advanced into the bore 12 by incrementally pushing the catheter 10 through tunneler 14.

Because the catheter 10 is relatively flexible, introduction of the catheter 10 to the tunneler in this manner is a difficult, time-intensive procedure which invites attempts to force the catheter through the tunneler 14. Improper handling of the catheter 10 in this manner can easily result in damage to the catheter 10 and/or catheter end 16. Presently, no system is available to facilitate the positioning of a catheter within a tunneler.

There is accordingly a need for a catheter feed through guide that facilitates the passage of a catheter through a tunneler and which avoids damage to the catheter and catheter ends. The present invention satisfies these needs, as well as others, and generally overcomes the deficiencies found in the background art.

### SUMMARY OF THE INVENTION

The present invention is a catheter feed through guide apparatus and method which facilitates the feeding of a catheter through a tunneler during implant procedures and avoids damage to the catheter and catheter tip during the catheter feed through procedure. In its most general terms, the catheter feed through apparatus of the invention comprises a guide element configured to fit through a tunneler and to releasibly engage a catheter or catheter end.

By way of example, and not necessarily of limitation, the elongated guide element comprises an elongated, resilient wire having first and second ends. The first end of the guide element is configured to releasibly couple to a catheter or catheter end and, preferably, a handle is included at the second end. The guide element is used in conjunction with an elongated, tubular tunneler, and the guide element has sufficient length with respect to the tunneler such that at least one of the ends of the guide element will always extend out of the tunneler when the guide element is positioned within the tunneler.

The first end of the guide element, certain embodiments, may comprise a helix of resilient material that is structured and configured to fit through the tunneler. The helix preferably has an internal diameter which is slightly less than the external diameter of the catheter which is to be introduced into the catheter. This allows the helix to tensionally engage the catheter when the catheter is positioned within the helix. The spacing between the individual coils of the helix is sufficient to allow the catheter to pass between the coils when the catheter is suitably positioned. The helix may also be made of resilient wire and may be integral to the guide element. Other means for releasibly coupling to a catheter may alternatively be provided in association with the guide element first end, such as a clamp, clip, hook, tensioner, pincer or other feature which can fit through a tunneler and releasibly engage, attach or otherwise couple to a catheter.

The handle, in its simplest form, may comprise a simple bead or protrusion on the end of the guide element to facilitate grasping and manipulation of the end of the guide element with forceps. The handle may alternatively be structured and configured to be manipulated by a user's hand.

The guide element of the invention allows a catheter to be quickly and easily drawn or guided through an elongated tunneler during an implant procedure. In the procedure, a first and second incisions are made in a patient, with one incision located generally at a drug delivery site, and the other incision made at a pump implant site. The tunneler is used to place the catheter under the patient's skin with the ends of the catheter positioned adjacent the incisions.

In operation of the invention, the helix at the first end of the guide element is coupled to a catheter adjacent to an end of the catheter by positioning the coils of the helix around the catheter so that the catheter is tensionally engaged by the coils of the helix. The handle of the guide element is inserted into a first end of the tunneler and the guide element is passed through the tunneler until the handle is adjacent to the opposite or second end of the tunneler. The second end of the tunneler is then inserted into the first incision and passed under the patient's skin until the second end of the tunneler reaches the second incision in the patient and the first end of the tunneler is adjacent to the first incision.

The handle on the guide element is then grasped, and the guide element is withdrawn from the tunneler second end, drawing the attached catheter with the guide element. When the helix clears the tunneler and the guide element is completely removed from the tunneler, the catheter is disengaged from the helix to leave the catheter extending through the tunneler, with one end of the catheter adjacent the first incision, and the opposite end (which was coupled to the helix) adjacent to the second incision. The catheter ends are held in place and the tunneler is removed from one of the incisions to leave the catheter correctly positioned under the patient's skin, with one end adjacent the first incision to be positioned for drug delivery to a desired drug delivery location, and with the other end positioned for coupling to an implantable pump.

In an alternate procedure, the end of the tunneler may first be inserted into the an incision and passed under the patient's skin until the tunneler is positioned with one adjacent to each of the incisions. The catheter end is coupled to the helix in the manner described above, and the handle on the guide element is inserted into a tunneler end, the guide element is passed through the tunneler in the manner described above, and the catheter is detached from the helix on the end of the guide element. The catheter ends are held in place and the tunneler removed via one of the incisions to leave the correctly positioned catheter.

In other variations of the above procedures, the helix at the first end of the guide element, together with the attached catheter, may be inserted into the first end of the tunneler and passed therethrough until the helix and attached catheter exit the second end of the tunneler. The catheter is then detached from the helix and used as described above.

The invention thus provides a method for guiding or feeding a catheter between first and second incisions in a patient, comprising coupling a catheter end to a guide element, passing the guide element through a tunneler associated with the first and second incisions, and detaching the catheter end from the guide element. The method also generally comprises inserting the tunneler into one of the incisions, and removing the tunneler from one of the incisions while leaving the catheter in place.

More specifically, the method of the invention comprises coupling a catheter end to a first end of a guide element, inserting a second end of the guide element into a first end of a tunneler, drawing the guide element through the tunneler, removing the guide element from a second end of the tunneler, and detaching the catheter end from the first end of the guide element. The method also comprises inserting the tunneler into one of the incisions and positioning the tunneler beneath the patient's skin.

Further objects and advantages of the invention will be made clear in the following detailed description.

### BRIEF DESCRIPTIONS OF THE DRAWINGS

The present invention will be more fully understood by reference to the following drawings, which are for illustrative purposes only.

FIG. 1 is a side view of a catheter and a tunneler in cross section which illustrates the prior art method of introducing a catheter into a tunneler.

FIG. 2 is a perspective view of a catheter feed through guide apparatus in accordance with the present invention.

FIG. 3 illustrates the coupling of the catheter feed through guide apparatus of FIG. 2 to a catheter.

FIG. 4 illustrates a catheter attached to a feed-through guide, being fed through a tunneler.

### DETAILED DESCRIPTION OF THE INVENTION

Referring more specifically to the drawings, for illustrative purposes the present invention is embodied in the apparatus and method shown generally in FIG. 2 through FIG. 4. It will be appreciated that the apparatus may vary as to configuration and as to details of the parts, and that the method may vary as to details and the order of the steps, without departing from the basic concepts as disclosed herein.

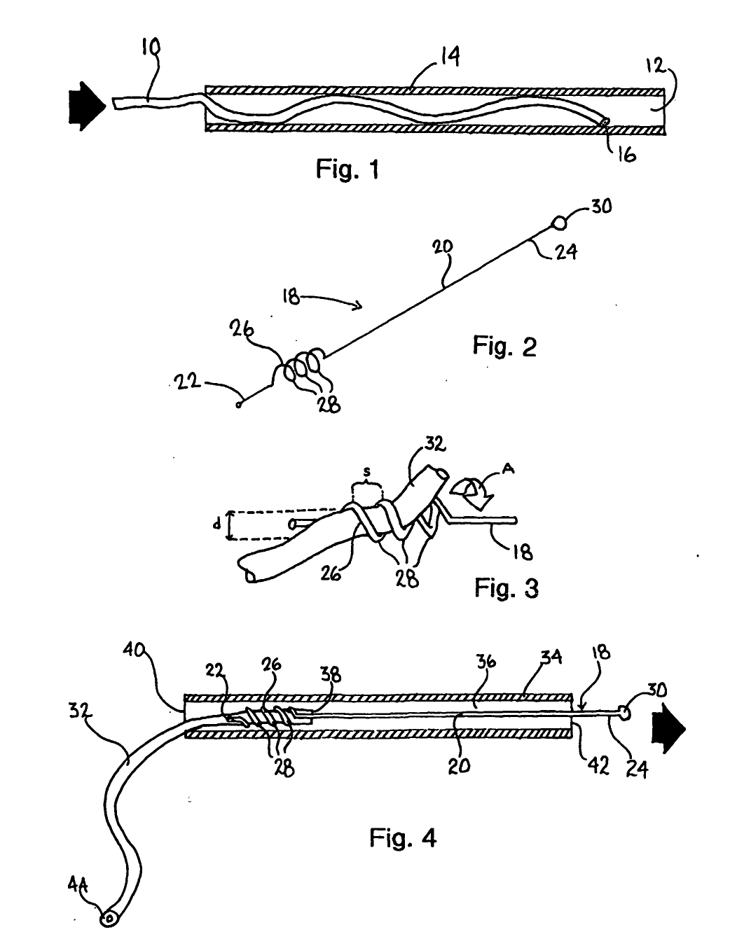

Referring now to FIG. 2, There is shown a catheter feed through guide apparatus 18 in accordance with the present invention. The apparatus 18 comprises an elongated guide element 20 with first and second ends 22, 24. In the embodiment shown in FIG. 1, a helix 26 is included on guide element 18 adjacent first end 22. Helix 26 includes a plurality of coils 28. A handle, shown as a simple bead or sphere 30, is included on guide element 18 adjacent second end 24.

Guide element 20 is preferably made of a resilient material such as a strip or wire of steel or like metal or metal alloy. Guide element 20 may alternatively be made of a resilient polymeric material. Helix 26 and handle 28 may be integral to guide element 20 and fabricated from the same material.

Referring also to FIG. 3, the helix 26 at first end 22 of guide element 18 is configured to releasibly couple to or engage a catheter 32. Catheter 32 generally comprises an elongated, flexible, elastomeric tube configured for delivery of therapeutic compositions as is well known in the art. Only a portion of catheter 32 is shown for reason of clarity. Helix 26 preferably has an inner diameter d which is slightly smaller than the outer diameter (not shown) of catheter 32, so that helix 26 will tensionally engage catheter 32 when catheter 32 is positioned within helix 26. The coils 28 of helix 26 are spaced apart from each other by a spacing distance s which is sufficient to allow catheter 32 to pass between adjacent coils 28 so that helix can be coupled or attached to, and then subsequently released from helix 26 as described further below.

Referring to FIG. 4 as well as FIG. 2 and FIG. 3, guide element 18 is structured and configured to fit or pass through a tunneler 34, and thus helix 26 and handle 30 are suitably dimensioned to allow helix 26 and handle 30 to fit through the bore 36 of tunneler. Tunneler 34 comprises an elongated, hollow, tubular structure of the type generally used for guiding or positioning catheters during implant surgery.

The dimensions tunneler 34, as well as those of the catheter feed through guide apparatus 18, and catheter 32, are not necessarily shown to scale in FIG. 2 through FIG. 4. In many implant surgeries, a tunneler 34 that is longer and thinner, or which otherwise has a greater aspect ratio than is shown, will be desirable. The particular length of and bore diameter of tunneler 34 will necessarily vary according to different surgical implant procedures, and the dimensions of the catheter feed through guide apparatus 18 will vary as well to accommodate the particular dimensions of tunneler 34. In certain embodiments, guide element 20 is sufficiently long such that at least one of the ends 22, 24 always extends out of, or is accessible via forceps from an end of tunneler 34, when guide element 20 is positioned within tunneler 34.

The catheter feed through guide apparatus 18 of the invention facilitates the positioning of catheter 32 within tunneler 34 for implant operations. In order to couple or attach catheter 32 to the apparatus 18, catheter 32 is positioned adjacent helix 26, and the catheter feed through guide apparatus 18 is rotated with respect to catheter 32 as shown by arrow A in FIG. 3. As the catheter feed through guide apparatus 18 is rotated, catheter 32 is flexed and pushed through the spacings s between coils 28 to position catheter 32 within helix 26, as shown in FIG. 4. As noted above, the inner diameter of helix 26 is slightly smaller than the outer diameter of catheter 32, so that the elastomeric material of catheter 32 is tensionally engaged by helix 26 to attach or couple catheter 32 to end 22 of the catheter feed through guide apparatus 18. As shown in FIG. 4, catheter 32 is coupled to end 22 of catheter feed through guide apparatus 18 adjacent to the end 38 of catheter 32. Helix 26 may alternatively be coupled to other portions of catheter 32.

When catheter 32 has been thus attached to catheter feed through guide apparatus 18, the handle 30 at end 24 of guide element 18 is inserted into a first end 40 of tunneler 34, and the catheter feed through guide apparatus 18 is pushed into and through bore 36 of tunneler 34 until handle 30 emerges from, or is adjacent to, the second end 42 of tunneler 34, as shown in FIG. 4. Handle 30 is then grasped, and the catheter feed through guide apparatus 18 is drawn through bore 36 of tunneler 34 so that helix 26 exits the second end 42 of tunneler 34. Catheter 30 is then released from helix 26 by rotating the catheter feed through guide apparatus 18 in the direction opposite to that use when coupling catheter 30 to helix 26, while flexing and positioning catheter 30 so that catheter 30 is removed from between the individual coils 28 of helix 26.

The use of the catheter feed through guide apparatus 18 in the above manner leaves catheter 32 positioned within tunneler 34, with end 44 of catheter 32 positioned adjacent end 40 of tunneler 34, and with end 38 of catheter 32 positioned adjacent end 42 of tunneler 34. Since the apparatus 18 can be quickly and easily passed through tunneler 34, the difficulty of incremental, manual feeding of a catheter through a tunneler is avoided.

The catheter feed through guide apparatus 18 of the invention simplifies catheter implant procedures. Catheter 32 is used generally to convey a drug or other therapeutic composition from an implanted pump or reservoir to a drug delivery site on a patient. In the implant procedure using the invention, first and second incisions (not shown) are made in a patient, with one incision located generally at the drug delivery site, and the other incision made at a pump implant site. The tunneler 34 is then inserted into one of the incisions and passed under the patients skin so that end 40 of tunneler 34 is positioned adjacent the first incision, and end 42 is positioned adjacent the second incision. Tunneler 34 thus defines a path under the patient's skin for placement of catheter 32.

The helix 26 at end 22 of the guide element 18 is coupled to catheter 32, and handle 30 on guide element 20 is inserted into end 40 of tunneler 34 and the catheter feed through guide apparatus 18 is passed through the tunneler 34 and removed from end 42 in the manner described above. The catheter feed through guide apparatus 18 is detached from catheter 32 to leaves catheter 32 positioned within tunneler 34 with end 44 of catheter 32 positioned adjacent end 40 of tunneler 34, and with end 38 of catheter 32 positioned adjacent end 42 of tunneler 34. The tunneler 34 is then withdrawn from one of the incisions while the catheter 32 is held in place to leave catheter 32 implanted beneath the patients skin with end 44 of catheter 32 adjacent one incision in the patient, and with end 38 of catheter 32 positioned adjacent the other incision. One of the catheter ends 38, 44 is then coupled to an implanted pump (not shown), and the other positioned to deliver drugs from the implanted pump, and the incisions are sutured closed in a conventional manner.

Several variations on the above procedure will suggest themselves to those skilled in the art, and are considered to be within the scope of the present invention. For example, the catheter feed through guide apparatus 18 and attached catheter 32 may be positioned within tunneler 34 prior to inserting tunneler 34 into a patient incision and positioning the tunneler beneath the patient's skin. Once the tunneler 34 is suitably positioned, the catheter feed through guide apparatus 18 is then removed from the tunneler 34 and detached from the catheter 32 as related above. In other procedures, the helix 26 and attached catheter 32 may be inserted into the tunneler 34 first, with the catheter feed through guide apparatus 18 pushed through tunneler 34 via handle until the helix 26 exits the opposite end of the tunneler 34. The catheter 32 would then be detached from the helix 26 and the catheter feed through guide apparatus 18 withdrawn from the tunneler 34 to leave the catheter 32 positioned within the tunneler 34.

The catheter feed through guide apparatus 18 may be configured differently for different uses of the invention. Helix 26 may, for example, be replaced with a different feature configured for releasibly coupling to a catheter, such as a clamp, clip, hook, tensioner, pincer or other feature which can fit through tunneler 34 and releasibly engage, attach or otherwise couple to catheter 32. Handle 30 is shown as a simple bead or protrusion on the end of the guide element 18 to facilitate grasping and manipulation of the end of the guide element with conventional forceps. Handle 30 may alternatively be structured and configured to be manipulated by a user's hand or a specific tool.

Accordingly, it will be seen that this invention provides a catheter feed through guide apparatus and method which facilitates the feeding of a catheter through a tunneler during implant procedures and avoids damage to the catheter and catheter tip during the catheter feed through procedure. Although the description above contains many specificities, these should not be construed as limiting the scope of the invention but as merely providing an illustration of the presently preferred embodiment of the invention. Thus the scope of this invention should be determined by the appended claims and their legal equivalents.

That which is claimed is:

1. A catheter guide apparatus comprising a guide element configured to fit through a tunneler and to releasibly engage a catheter.

2. The catheter guide apparatus of claim 1, wherein said guide element further comprises first and second ends, said first end configured to releasibly couple to said catheter.

3. The catheter guide apparatus of claim 2, wherein said guide element has a length sufficient such that at least one of said first and second ends extends out of said tunneler when said guide element is positioned within said tunneler.

4. The catheter guide apparatus of claim 1, wherein said guide element further comprises a helix adjacent one end, said helix including at least two coils, said spacing between said coils being sufficient to pass said catheter between adjacent ones of said coils.

5. The catheter guide apparatus of claim 4, wherein said helix has an internal diameter which is slightly smaller than an external diameter of said catheter.

6. The catheter guide apparatus of claim 2, wherein said second end includes a handle.

7. A catheter feed through guide apparatus comprising an elongated, resilient guide element having first and second ends, said first end configured to releasibly couple to a catheter, said elongated, resilient guide element configured to fit through a tunneler.

8. A catheter feed through guide apparatus of claim 7, wherein said elongated, resilient guide element has a length sufficient such that at least one of said first and second ends extends out of said tunneler when said guide element is positioned within said tunneler.

8. A catheter feed through guide apparatus of claim 7, wherein said first end includes a helix having a plurality of coils, said spacing between said coils being sufficient to pass said catheter between adjacent ones of said coils.

9. The catheter feed through guide apparatus of claim 8, wherein said helix has an internal diameter which is slightly smaller than an external diameter of said catheter;

10. A method for feeding a catheter through a tunneler, comprising attaching said catheter to a guide element, and passing said guide element and attached said catheter through said guide tube.

11. The method of claim 10, further comprising detaching said catheter from said guide element after passing said guide element and attached said catheter through said tunneler.

12. A method for positioning a catheter between first and second incisions in a patient, comprising:
(a) attaching a catheter to a guide element;
(b) passing said guide element through a tunneler associated with said first and second incisions; and
(c) detaching said catheter from said guide element.

13. The method of claim 12, further comprising:
(a) inserting said tunneler into one of said incisions and positioning said tunneler beneath said patient's skin between said first and second incisions; and
(b) after passing said guide element through said tunneler and detaching said catheter therefrom, removing said tunneler from one of said incisions while leaving said catheter in place beneath said patient's said skin.

14. A method for guiding a catheter through a tunneler, comprising:
(a) coupling a catheter to a first end of a guide element;
(b) inserting a second end of said guide element into a first end of a tunneler;
(c) drawing said guide element through said tunneler and removing said guide element from a second end of said tunneler, and
(d) detaching said catheter from said first end of said guide element.
